(19) United States
(12) Patent Application Publication
(10) Pub. No.: US 2026/0076633 A1
(43) Pub. Date: Mar. 19, 2026

(54) COMPENSATED MAMMOGRAPHY WITH COMPRESSION PLATE CONTAINING BIOPSY APERTURE

(71) Applicant: Siemens Healthineers AG, Forchheim (DE)

(72) Inventors: Christopher SYBEN, Cadolzburg (DE); Ferdinand LUECK, Erlangen (DE); Marcel BEISTER, Erlangen (DE); Ludwig RITSCHL, Buttenheim (DE)

(73) Assignee: Siemens Healthineers AG, Forchheim (DE)

(21) Appl. No.: 19/329,966

(22) Filed: Sep. 16, 2025

(30) Foreign Application Priority Data
Sep. 17, 2024 (DE) 10 2024 208 854.4

(51) Int. Cl. A61B 6/46; A61B 6/00; A61B 6/04; A61B 6/50; A61B 6/58; G06T 5/60; G06T 5/77; G06T 7/11

(52) U.S. Cl. CPC A61B 6/463; A61B 6/0414; A61B 6/0435; A61B 6/502; A61B 6/5211; A61B 6/582; G06T 5/60; G06T 5/77; G06T 7/11; G06T 2207/10012; G06T 2207/10112; G06T 2207/10116; G06T 2207/20084; G06T 2207/30068

(57) ABSTRACT

A method for image processing of mammography image data from a mammography system, comprises: acquiring mammography image data of a breast of a patient; and determining corrected mammography image data based on the mammography image data, wherein the corrected mammography image data is modified to at least partially compensate for the influence of the cutout on the representation of the breast in the mammography image data.

### CROSS-REFERENCE TO RELATED APPLICATION(S)

The present application claims priority under 35 U.S.C. § 119 to German Patent Application No. 10 2024 208 854.4, filed Sep. 17, 2024, the entire contents of which is incorporated herein by reference.

### FIELD

One or more example embodiments of the present invention relate to a method for image processing of mammography image data from a mammography system having a compression plate containing a cutout. One or more example embodiments of the present invention also relate to an image data processing device. In addition, one or more example embodiments of the present invention relate to a mammography system.

### BACKGROUND

In a breast examination, breast imaging is in many cases combined with a biopsy of the breast of a patient in order to be able to establish and determine by the breast imaging the position of a biopsy needle being used for the biopsy. Contrast agents, which produce a contrast with surrounding regions in spectral imaging, are used to reveal the lesions to be examined in the breast. Before a mammography acquisition, a compression plate is used to press and strongly compress in the vertical direction the breast of the patient against a breast support.

For a biopsy, initially a scout image is acquired of the compressed breast to be examined in order to check whether the breast has been positioned correctly for the subsequent biopsy. In addition, 3D imaging can take place in order to verify the positioning of the biopsy needle. Such 3D imaging can be performed by stereo imaging or tomosynthesis. After a tissue sample has been taken, further imaging of the breast can take place in order to determine whether the tissue sample was removed correctly.

There are two different ways to proceed in order to reach a lesion with a biopsy needle. The biopsy needle can be inserted into the breast from the side or from above. If a lateral biopsy, i.e. a biopsy from the side, is being carried out, the breast can be compressed by a closed paddle or closed compression plate. If, however, access to the breast for the biopsy is from above, an aperture or cutout needs to be provided in the compression plate in order to make the lesion accessible to the biopsy needle from above.

In practice, acquisitions are often made using the open compression plate because this means that both types of access are possible and the type of access can be chosen flexibly on the basis of the image data. Thus the problem of imaging in which the cutout is also shown in the image can arise with both lateral and vertical access for the biopsy.

This cutout leads to two effects in the image data acquired for the biopsy:
since no attenuation of the X-ray radiation as a result of the compression plate occurs in the region in which the aperture in the compression plate is present, the aperture is clearly visible in the acquired image data. Thus there is an image region in which attenuation exists which is reduced by an offset and hence also a grayscale value of reduced brightness compared with the rest of the image;
the compression force causes the breast tissue to bulge into the cutout in the compression plate. This change results in a different compression of the breast tissue in this region, producing in this region an additional change in the grayscale values but which is curved in shape because the breast tissue is also rounded. This leads to an increased attenuation, i.e. results in grayscale values of greater brightness or reduced (dark) intensity of the grayscale values compared with a depiction without this bulging of the breast tissue.

When the term attenuation is used, it is intended to mean the attenuation of the X-ray radiation, which attenuation is caused by the material through which the X-ray radiation passes. The grayscale values, on the other hand, are stronger, or darker, the more X-ray radiation falls onto the X-ray detector, i.e. the lower the attenuation. Conversely, grayscale values are brighter, the stronger the attenuation, because in this case only a little X-ray radiation falls onto the X-ray detector and therefore no reduction in the brightness of the detector signals takes place, analogous to the absence of blackness on an X-ray plate.

Usually, the acquired images are processed such that the breast tissue in the field of view is displayed in a grayscale window so that the radiologist can examine the whole image without having to adjust grayscale levels. Unfortunately, the two phenomena described above lead to a far wider range of grayscale values in the image data. This limits the possibility of suitable visualization. In addition, the offset and the intensity change resulting from the protrusion of the breast in the region of the cutout in the compression plate alter the brightness of the lesions revealed by the contrast. Furthermore, brightness values of tissue enhanced by contrast agent are altered compared with tissue and lesions outside the cutout in the compression plate, making it difficult to assess and compare the contrast-agent enhancement.

Without compensation, the images are shown with the cutout in the compression plate and with the protrusion of the breast into the cutout.

### SUMMARY

An object of one or more example embodiments of the present invention is to develop a method and an apparatus for image processing which compensate an artifact-like image effect caused by the cutout in the compression plate. Preferably, at least one of the aforementioned image effects shall be compensated, particularly preferably both image effects shall be compensated.

At least this object is achieved by a method for image processing of mammography image data from a mammography system having a compression plate containing a cutout, by an image data processing device, and by a mammography system as claimed.

In the method, according to one or more example embodiments of the present invention, for image processing of mammography image data from a mammography system having a compression plate containing a cutout, preferably with vertical access for a biopsy needle, mammography image data is acquired from a breast of a patient. Mammography image data shall be understood to mean image data from the breast of a female patient, or in individual cases of a male patient, which is obtained by X-ray imaging and is used in particular for early detection of breast cancer or its precursors. The cutout is preferably located in a center region of the compression plate, particularly preferably in a central region of the compression plate.

Mammography image data comprises in particular image data from the breast of the following type:
image data that is acquired from a central position at an angle of 0°;
stereo image data;
tomosynthesis image data;
full views;
scout images.

Image data that is acquired from a central position at an angle of 0°represents classic mammography image data acquired “from above”or in a vertical direction.

Stereo image data comprises images acquired from different angles, in particular from 25° and +25°.

Scout images with reduced resolution and reduced image segment are particularly suitable for ascertaining and monitoring the position of biopsy needles in the breast tissue.

In principle, the method according to one or more example embodiments of the present invention can also be used to produce full views of the breast with high resolution and an entire image region.

In addition, corrected mammography image data can be determined on the basis of the acquired mammography image data, wherein image effects of the cutout are determined in the acquired mammography image data, and the mammography image data is modified in such a way that the image effects are at least partially compensated.

Thus corrected mammography image data is determined on the basis of the acquired mammography image data, wherein the corrected mammography image data is modified in such a way that the influence of the cutout on the representation of the breast in the mammography image data is at least partially compensated. As already mentioned, during the imaging the X-rays fall vertically through the cutout onto the breast and, because of the lack of compression-plate material there, are attenuated less in the region of the cutout than outside this region. In addition, the breast protrudes into the cutout, which likewise leads to an unwanted change in the image representation.

Advantageously, at least some of the image effects, preferably all the image effects of the vertical access of the mammography system or the cutout are compensated, and a visual impression is produced that is comparable to the image data from a mammography system having a closed compression plate. The intensity values or grayscale values of the image data can be compared, regardless of whether they lie inside or outside the region of the cutout for the biopsy needle or whether they lie at certain points in this region. For example, without compensation, the grayscale values in the center of the cutout can be darker because of a cutout than at the edge of the cutout, because the attenuation of the X-ray radiation is reduced as a result of the cutout. This can cause the lesions revealed by iodine to appear darker in the region of the cutout compared with lesions outside the region of the cutout, although they would be brighter without the cutout. Since the radiologist usually looks for lesions of greatest brightness, this might induce him to make an incorrect repositioning, which is avoided by the correction based on the method according to one or more example embodiments of the present invention. The radiologist is able to distinguish iodine from dense glandular tissue by acquiring two images using two different energy values and setting one off against the other.

The image data processing device according to one or more example embodiments of the present invention comprises an input interface for receiving acquired mammography image data from a breast of a patient. In addition, the image data processing device according to one or more example embodiments of the present invention has a correction unit for determining corrected mammography image data on the basis of the acquired mammography image data, wherein image effects of the cutout, which preferably relate to a change in attenuation values in the mammography image data, are determined in the acquired mammography image data, and the mammography image data is modified in such a way that the image effects are at least partially compensated.

The corrected mammography image data is altered in such a way that the influence of the cutout on the representation of the breast in the mammography image data is at least partially compensated. The image data processing device according to one or more example embodiments of the present invention shares the advantages of the method according to one or more example embodiments of the present invention for image processing of mammography data from a mammography system having vertical access for a biopsy needle.

The mammography system according to one or more example embodiments of the present invention has a scanner unit having a compression plate containing a cutout, preferably with vertical access for a biopsy needle, for acquiring mammography image data from the breast of a patient. In addition, the mammography system according to one or more example embodiments of the present invention comprises a control device for controlling the scanner unit for mammography imaging and an image data processing device according to one or more example embodiments of the present invention. The mammography system according to one or more example embodiments of the present invention shares the advantages of the image data processing device according to one or more example embodiments of the present invention.

Most of the aforementioned components of the image data processing device according to one or more example embodiments of the present invention can be implemented in full or in part in the form of software modules in a processor of a suitable computing system, for example by a control device of a mammography system or by a computer used to control such a system. An implementation largely in software has the advantage that even computing systems already in use can be easily upgraded by a software update in order to work in the manner according to one or more example embodiments of the present invention.

In this respect, the object is also achieved by a corresponding non-transitory computer program product having a computer program, which can be loaded directly into a computing system and which contains program segments in order to perform the steps of the method according to one or more example embodiments of the present invention for image processing of mammography image data from a mammography system having a compression plate containing a cutout when the program is executed in the computing system. Said computer program product may comprise in addition to the computer program, if applicable, extra elements such as e.g. documentation and/or extra components, including hardware components, such as e.g. hardware keys (dongles etc.) for using the software.

For transfer to the computing system and/or for storage on, or in, the computing system, a non-transitory computer-readable medium, for instance a memory stick, a hard disk or any other portable or permanently installed data storage medium can be used, on which are stored the program segments of the computer program, which program segments can be read in and executed by a computing system. For this purpose, the computing system can comprise, for example, one or more interacting microprocessors or the like.

The dependent claims and the following description each contain particularly advantageous embodiments and developments of the present invention. In particular, the claims in one category of claims can also be developed in a similar way to the dependent claims in another category of claims. Furthermore, within the scope of the present invention, the various features of different exemplary embodiments and claims can also be combined to create new exemplary embodiments.

In a preferred variant of the method according to one or more example embodiments of the present invention, the determining of the corrected mammography image data comprises the sub-steps:
determining the position and/or extent of the cutout in the mammography image data on the basis of the mammography image data;
determining corrected mammography image data on the basis of the determined position and/or the extent of the cutout in the mammography image data.

The position and/or extent of the cutout in the mammography image data shall be understood to mean in this context a subregion of the imaged breast in the mammography image data, which subregion is exposed to, and penetrated by, the X-rays that reach this subregion through the cutout.

Advantageously, the region affected by the cutout or the radiation through the cutout, is thus first localized in the mammography image data. In a second step, the localized consequences of this cutout are analyzed and compensated in terms of image effects that arise.

In the compensation, the corrected mammography image data is preferably modified in such a way that a lack of attenuation caused by a lack of paddle material or lack of compression-plate material in the region of the cutout is at least reduced in the mammography image data on the basis of the determined position and extent of the cutout in the mammography image data.

The analysis of the lack of attenuation preferably exploits the fact that the lack of attenuation caused by a lack of compression-plate material in the region of the cutout has a constant offset. In the determining of the corrected mammography image data, the constant offset is then compensated, and preferably the constant offset is eliminated by the grayscale values in the mammography image data at the edge of the cutout adopting the same value outside and inside the cutout.

For determining the offset first arithmetic mean or median values of grayscale values outside and inside, the cutout can be determined, in particular at the border or edge of the cutout or of the respective regions corresponding to the border of the cutout in the mammography image data, and then difference values between grayscale values or attenuation values outside and inside the cutout can be calculated row by row or column by column in the border region. The constant offset can then be made by calculating the median on the basis of the determined difference values. Alternatively, the offset can also be determined by an artificial neural network.

Alternatively, the offset can also be ascertained in advance by calibration and retrieved, for example, from a stored table.

Preferably, the determining of the position and/or extent of the cutout in the mammography image data on the basis of the mammography image data comprises segmentation of the mammography image data, wherein the region of the extent of the cutout in the mammography image data is segmented. Advantageously, the cutout, or the region in the mammography image data that is exposed to the X-ray radiation falling onto the breast through the cutout, can be demarcated with respect to the surroundings. A border region or transition region can thereby be defined that can be used to determine the above-mentioned difference values for calculating the offset, and can also be used as a demarcation for further corrections.

The segmentation is performed here on the basis of one of the following methods:
a thresholding method, in particular what is known as Otsu's thresholding;
an artificial neural network, trained for segmentation, preferably a U-net.

A plausibility check can then be made on the results of both methods, because although both the size and position of the cutout is normally known with less accuracy than when determined in the method according to one or more example embodiments of the present invention, this less accurate data can be used as comparative values. If a large discrepancy arises in the comparison, this indicates that an error has occurred in applying the method according to one or more example embodiments of the present invention.

Thresholding methods comprise a group of algorithms for segmenting digital image data. Segmentation can be an important step for image analysis, for instance in order to recognize objects in the image. Thresholding methods can be used to decide in simple situations which picture elements represent objects being sought and which belong to their surroundings. Thresholding methods result in binary images. The motivation for using binary images is usually the availability of fast binary-image algorithms.

As in all segmentation methods, it is also the case in the thresholding methods that picture elements-also known as pixels-are assigned to what are known as the segments. The image to be segmented is here in the form of numerical values (one or more color values per pixel). The comparison of the grayscale value or another one-dimensional feature with a threshold value decides whether a pixel belongs to a segment. The grayscale value of a pixel is purely a brightness value; additional color information is not taken into account. Since this operation is usually applied independently for each pixel, the thresholding method is what is known as a pixel-oriented segmentation method

Thresholding methods belong to the oldest methods in digital image processing. Otsu's method as an example variant of such a thresholding method was published in 1979 by Nobuyuki Otsu. Thanks to their simplicity, thresholding methods can be implemented quickly, and segmentation results can be calculated with a small amount of effort.

The cited thresholding method by Otsu is described in Nobuyuki Otsu: A threshold selection method from grey level histograms, in: IEEE Transactions on Systems, Man, and Cybernetics. New York, 9.1979, p. 62-66. ISSN 1083-4419.

Segmentation using a trained artificial neural network, in particular a U-net, has the advantage of being highly flexible to apply to objects or sub-regions to be segmented. A U-net is an artificial convolutional neural network. The network is based on a fully convolutional neural network, the architecture of which has been modified and extended to work with fewer training images and to allow precise segmentation.

The mammography image data preferably comprises spectral image data containing low-energy image data and high-energy image data, and the segmentation in mammography image data in image data of one of the following image data types:
in the low-energy image data;
in the high-energy image data;
in spectrally recombined image data.

The choice of the image data types for segmentation and compensation depends on the nature of the recombination and of the image processing algorithms that are to be used in imaging. If segmentation is performed in the low-energy image data and in the high-energy image data and also in the spectrally recombined image data, then this increases the flexibility in the use of the image data or the image processing.

For example, the bulging of the breast produces a thickness change in the region of the cutout in the compression plate. Since, however, the recombination algorithm uses the thickness for the recombination, segmentation just in the low-energy image data or the high-energy image data can result in a mask for which the thickness of the breast tissue behaves differently than in the fully compressed region outside the region of the cutout.

Segmentation in the low-energy image data or high-energy image data allows, on the basis of the method according to one or more example embodiments of the present invention, a thickness of the breast tissue to be corrected. Since the thickness is needed for correct recombination, however, it is advantageous from this perspective to perform the segmentation in the low-energy image data or high-energy image data.

On the other hand, the segmentation also makes sense in spectrally recombined image data, because in this case the contrast of the cutout compared with the surroundings is particularly strong, and hence segmentation can be performed particularly precisely.

In an embodiment of the method according to one or more example embodiments of the present invention, the lack of attenuation caused by a lack of compression-plate material in the region of the cutout is determined on the basis of segmentation, and in the segmented region of the cutout on the basis of applying a trained artificial neural network to the mammography image data of the cutout. In this variant, segmentation takes place first and then an artificial neural network is used to determine the change in the grayscale values. Advantageously, access can be made to an interim prediction of the method according to one or more example embodiments of the present invention. In this case, the segmentation can be checked to determine whether it is correct. Advantageously, in comparison with using a holistic image-to-image network, individual process steps can be supervised and, if applicable, modified, during the training of the network.

The determining of the lack of attenuation caused by a lack of compression-plate material in the region of the cutout can also be performed directly by a trained holistic artificial neural network, with unsegmented mammography image data used as the input data. Advantageously, just a method for processing the image data is needed. This variant can dispense with explicit segmentation and explicit determination of an offset. This simplifies the generation of training data because segmented image data does not have to be generated.

The corrected mammography image data is preferably modified in the manner that a lack of attenuation caused by a convex distension or protrusion of the breast into the region of the extent of the cutout is at least partially compensated. Advantageously, not only is a constant offset in a difference in the grayscale values of the image data compensated, but also locally location-dependent differences in the grayscale values that result from a deformation of the breast caused by the arrangement of the cutout are canceled out. The corrected depiction of the breast is thus made as though the cutout in the compression plate were not present and therefore the protrusion would also not occur.

It is likewise preferred to determine first the region of the extent of the cutout by segmentation, and to determine the lack of attenuation resulting from the convex distension of the breast by one of the following methods:
a function curve which is adapted to the grayscale values of the mammography image data in the region of the extent of the cutout;
an artificial neural network.

A function curve shall be understood to mean here a location-dependent function, where the location lies on a two-dimensional surface. The function curve shall be parameterizable depending on the acquired mammography image data. The function curve preferably comprises a polynomial or what is known as a spline, each of which has parameters, the associated parameter values of which are fitted to the grayscale values of the acquired mammography image data in the region of the cutout. A spline is a function composed piecewise of polynomials of order n. On the basis of the acquired mammography image data, interpolation points are defined for the function curve that are spanned by the function curve. In such a procedure in which a fixed model is used, no training data is needed to achieve compensation of image effects resulting from the protrusion of the breast into the cutout. The function is inversely applied to the grayscale values in order to obtain the correct shape of the protrusion. Since the cutout occurs in a planar region, a 2D polynomial or a 2D spline would be used for the mapping. A 2nd order 2D polynomial would have six free parameters, for example.

On the other hand, an artificial neural network is particularly flexible to use and, if applicable, given suitable training, can also take better account of contrasts of lesions than in the fixed model approach, in which potential lesions are used erroneously as the interpolation points for adapting the function curve.

The segmentation of the region of the cutout, the compensation of the image effect that this produces directly, and the compensation of the protrusion are preferably determined by separate algorithms that are executed successively. Advantageously, in this variant, the interim results are visible and also individual components of the compensation can be enabled and disabled. For example, just segmentation can be performed so that a user recognizes an image region in which potentially unreliable image data exists. If required, the above-described different compensations, i.e. the compensation of the lack of attenuation caused by the cutout and the compensation of the protrusion of the breast into the region of the cutout can then be performed individually as needed in order to obtain a corrected image.

### BRIEF DESCRIPTION OF THE DRAWINGS

The present invention is described again below in greater detail using exemplary embodiments and with reference to the accompanying figures, in which:

FIG. 1 shows a schematic representation of mammography image data viewed from above;

FIG. 2 shows a flow diagram illustrating a method for image processing of mammography image data from a mammography system having a compression plate containing a cutout with vertical access for a biopsy needle according to a first exemplary embodiment of the present invention;

FIG. 3 shows a flow diagram illustrating a method for image processing of mammography image data from a mammography system having a compression plate containing a cutout with vertical access for a biopsy needle according to a second exemplary embodiment of the present invention;

FIG. 4 shows a flow diagram illustrating a method for image processing of mammography image data from a mammography system having a compression plate containing a cutout with vertical access for a biopsy needle according to a third exemplary embodiment of the present invention;

FIG. 5 shows a flow diagram illustrating a method for image processing of mammography image data from a mammography system having a compression plate containing a cutout with vertical access for a biopsy needle according to a fourth exemplary embodiment of the present invention;

FIG. 6 shows a schematic representation of an image data processing device according to an exemplary embodiment of the present invention;

FIG. 7 shows a schematic representation of a mammography system according to an exemplary embodiment of the present invention.

### DETAILED DESCRIPTION

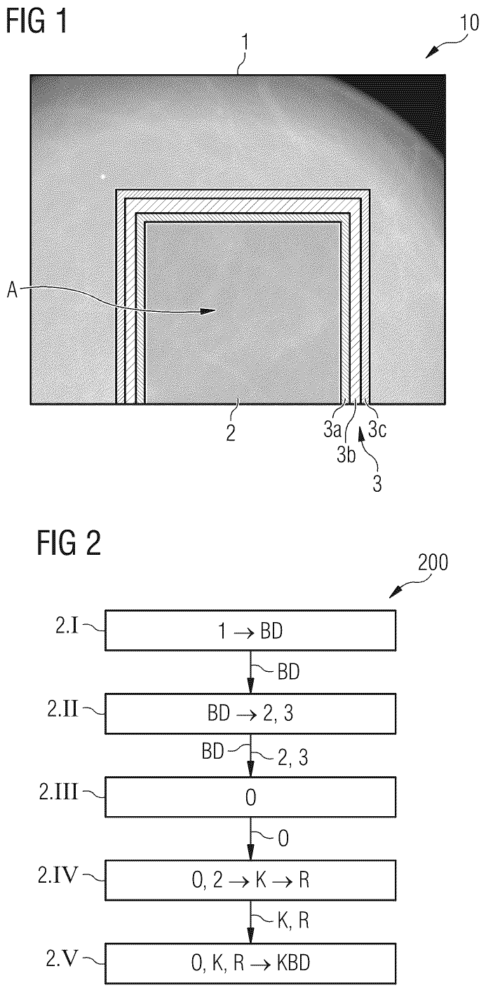

FIG. 1 shows a schematic representation 10 of mammography image data from a breast 1 of a patient viewed from above. In the center is shown a region 2 of a cutout A of a compression plate. Between the cutout A and an outside region there is a border region 3 having an inner border region 3a, an intermediate region 3b, and an outer border region 3c.

The region 2 of the cutout A is determined in a segmentation process. In the border region 3 a step-like transition of the grayscale values between the region 2 of the cutout A and the outside region is expected. For example, the grayscale values in the region of the outer border region 3c should turn out to be lower than the grayscale values in the inner border region 3a by an offset.

FIG. 2 shows a flow diagram 200 illustrating a method for image processing of mammography image data BD from a mammography system 70 (see FIG. 7) having a compression plate containing a cutout A with vertical access for a biopsy needle according to a first exemplary embodiment of the present invention.

In step 2.I, mammography image data BD is acquired from a breast 1 of a patient. The acquisition takes place during a vertical X-ray image acquisition of the breast 1 of the patient. As a result of the cutout A present in the compression plate of the mammography system 70, a region 2, which represents the cutout A, is visible in the mammography image data BD.

In step 2.II, the region 2 of the cutout A is localized in the mammography image data BD on the basis of the acquired mammography image data BD. In particular, the border region 3 already illustrated in FIG. 1 is determined and localized. This localization is performed by segmentation. In the exemplary embodiment illustrated in FIG. 2, the segmentation is performed via an Otsu thresholding method.

In step 2.III, first a constant offset O or offset value is determined, which occurs in the segmented region 2 because of the non-existent absorption there of X-ray radiation by the compression plate. The determining of the offset value can be determined from the different grayscale values in the inner and the outer border regions 3a, 3c (see FIG. 1) of the segmented region 2 of the cutout A by obtaining a mean value of the grayscale values for both the inner and the outer border regions 3a, 3c, and determining the difference between the two mean values as the offset value.

In step 2.IV, a curved reduction R in the attenuation resulting from protrusion of the breast into the region 2 of the cutout is determined by fitting a polynomial-based curve K to attenuation values in this region. The attenuation resulting from the reduced compression is strongest where the protruding is greatest. The fitting of the polynomial is performed by a regression method, which is applied to the attenuation values of the mammography image data BD and the known offset O. On the basis of the polynomial or the curve K determined therefrom, and the locally location-dependent reduction R in the grayscale values which is derived therefrom, the grayscale values in the segmented cutout are now corrected, resulting in a distribution of the grayscale values that would be obtained if there were no cutout in the compression plate.

In step 2.V, corrected mammography image data KBD is determined based on the knowledge of the offset O and the curve K and the locally location-dependent reduction R in the grayscale values that is determined therefrom, and is output.

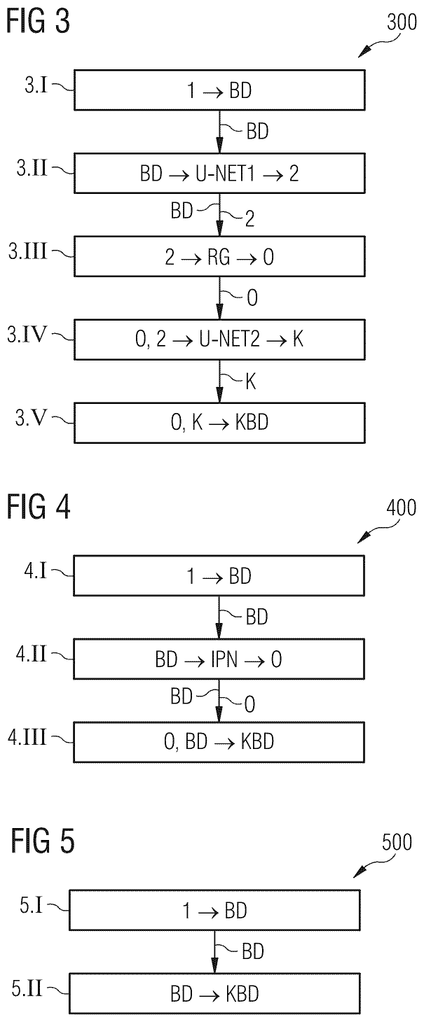

FIG. 3 shows a flow diagram 300 illustrating a method for image processing of mammography image data BD from a mammography system 70 (see FIG. 7) having a compression plate containing a cutout with vertical access for a biopsy needle according to a second exemplary embodiment of the present invention.

In step 3.I, mammography image data BD is acquired from a breast 1 of a patient. The acquisition takes place during a vertical X-ray image acquisition of the breast 1 of the patient. As a result of the cutout A present in the compression plate of the mammography system, a region 2, which represents the cutout A, is visible in the mammography image data BD.

In step 3.II, localization of the region 2 of the cutout A in the mammography image data BD on the basis of the acquired mammography image data BD is now performed by applying a first trained artificial neural U-network U-NET1.

In step 3.III, by applying a regression method RG, an offset value O is determined, which is used to compensate a change in the grayscale values in the segmented region.

In step 3.IV, a curved reduction R in the attenuation resulting from protrusion of the breast into the region 2 of the cutout A is determined by fitting a polynomial-based curve K to grayscale values in this region. In this exemplary embodiment, however, unlike the exemplary embodiment illustrated in FIG. 2, the fitting is not performed by a regression method but by applying a second trained artificial neural network U-NET2, in this case a U-network, to the segmented mammography image data BD. In particular, the second artificial neural network U-NET2 can learn to ignore certain image regions enhanced by iodine inside the cutout and to prevent a reduction in the contrast of the lesions.

In step 3.V, corrected mammography image data KBD is determined based on the knowledge of the offset O and the curve K, and is output.

FIG. 4 shows a flow diagram 400 illustrating a method for image processing of mammography image data BD from a mammography system 70 (see FIG. 7) having a compression plate containing a cutout A with vertical access for a biopsy needle according to a third exemplary embodiment of the present invention.

In step 4.I, mammography image data BD is acquired from a breast 1 of a patient. The acquisition takes place during a vertical X-ray image acquisition of the breast 1 of the patient. As a result of the cutout A present in the compression plate of the mammography system 70, a region 2 (see FIG. 1), which represents the cutout A, is visible in the mammography image data BD.

Applied in step 4.II is what is known as an inpainting network IPN as a variant of a trained artificial neural image-to-image network, in which the offset value O produced as a result of the cutout A is fully compensated in the region 2 of the cutout A in the compression plate. Thus in this variant, the steps of segmentation and of determining the offset value O are performed in a joint step by applying the artificial neural network IPN mentioned.

In step 4.III, direct compensation of the grayscale values corrupted by protrusion of the breast is now performed by a trained image-to-image network, without a polynomial being determined and in which corrected mammography image data KBD is again obtained. Thus figuratively speaking, steps 3.IV and 3.V are combined.

FIG. 5 shows a flow diagram 500 illustrating a method for image processing of mammography image data BD from a mammography system 70 having a compression plate containing a cutout A with vertical access for a biopsy needle according to a fourth exemplary embodiment of the present invention.

In step 5.I, mammography image data BD is acquired from a breast 1 of a patient. The acquisition takes place during a vertical X-ray image acquisition of the breast 1 of the patient. As a result of the cutout A present in the compression plate of the mammography system, a region 2, which represents the cutout A, is visible in the mammography image data BD.

In step 5.II. a holistic trained artificial neural network is now applied to the acquired mammography image data BD, and the already explained steps of segmentation, compensating for the cutout in the compression plate and compensating for the protruding of the breast into the region of the cutout A in the compression plate are performed in a shared step in which corrected mammography image data KBD is produced.

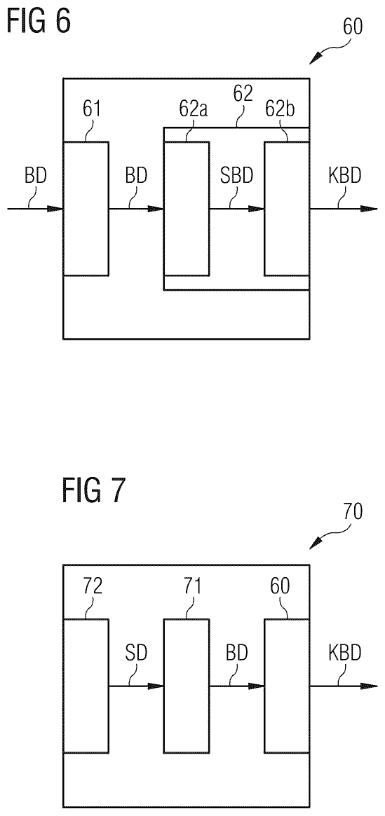

FIG. 6 shows a schematic diagram of an image data processing device 60 according to an exemplary embodiment of the present invention.

The image data processing device 60 has an input interface 61, which is configured to receive acquired mammography image data BD from a breast 1 of a patient.

The image data processing device 60 has a correction unit 62 for determining corrected mammography image data KBD on the basis of the acquired mammography image data BD, wherein the corrected mammography image data KBD is modified in such a way that the influence of the cutout A on the representation of the breast 1 in the mammography image data BD is compensated.

Part of the correction unit 62 is a determination unit 62a, which is configured to determine segmented mammography image data SBD on the basis of the mammography image data BD, in which a region 2 that has been exposed to radiation through a cutout A is localized.

The correction unit 62 also has a compensation unit 62b, which is configured to determine corrected mammography image data KBD on the basis of the segmented mammography image data SBD. The compensation unit 62b has the function of determining an offset O, which is used to cancel out the lack of attenuation in the X-ray radiation in the region of the cutout in the compression plate. In addition, the compensation unit 62b has a function for compensating protrusion of the breast 1 into this cutout. As already mentioned in connection with FIG. 2 to FIG. 5, the functions can comprise classical algorithms or be based on artificial intelligence.

FIG. 7 shows a schematic diagram of a mammography system 70 according to an exemplary embodiment of the present invention.

The mammography system 70 has a scanner unit 71 (shown in FIG. 7 in the center of the drawing) having a compression plate (not shown) containing a cutout having vertical access for a biopsy needle for acquiring mammography image data from the breast 1 of a patient.

Part of the mammography system 70 is also a control device 72 for controlling the scanner unit 71 via control data SD for the purpose of acquiring mammography image data BD. The mammography system 70 also comprises the image data processing device 60, which is illustrated in FIG. 6, for generating corrected mammography image data KBD on the basis of the acquired mammography image data BD.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of”has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled. ” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,”versus “directly between,”“adjacent,”versus “directly adjacent,”etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example”is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Finally, it shall be reiterated that the methods and devices described above are merely preferred exemplary embodiments of the present invention, and that the present invention can be modified by a person skilled in the art without departing from the scope of the present invention insofar as this is defined by the claims. It is mentioned for the sake of completeness that the use of the indefinite article “a” or “an” does not rule out the possibility of there also being more than one of the features concerned. Likewise, the term “unit” does not exclude the possibility that said unit consists of a plurality of components, which may also be spatially distributed if applicable. Independent of the grammatical term usage, individuals with male, female or other gender identities are included within the term.

What is claimed is:

1. A method for image processing of mammography image data from a mammography system having a compression plate containing a cutout, the method comprising:
acquiring mammography image data of a breast of a patient; and
determining corrected mammography image data based on the mammography image data, wherein image effects of the cutout are determined in the mammography image data, and the mammography image data is modified to at least partially compensate for the image effects.

2. The method as claimed in claim 1, wherein the mammography image data includes at least one of
image data that is acquired from a central position at an angle of 0°,
stereo image data,
tomosynthesis image data,
full views, or
scout image data.

3. The method as claimed in claim 1, wherein the determining of the corrected mammography image data comprises:
determining at least one of a position or an extent of the cutout in the mammography image data based on the mammography image data; and
determining the corrected mammography image data based on at least one of the position or the extent of the cutout in the mammography image data.

4. The method as claimed in claim 3, wherein the corrected mammography image data is modified such that a lack of attenuation caused by a lack of compression-plate material in a region of the cutout is at least reduced in the mammography image data based on at least one of the position or the extent of the cutout in the mammography image data.

5. The method as claimed in claim 4, wherein
the lack of attenuation caused by the lack of compression-plate material in the region of the cutout has a constant offset, and
the constant offset is compensated in determining the corrected mammography image data.

6. The method as claimed in claim 3, wherein the determining of at least one of the position or the extent of the cutout in the mammography image data based on the mammography image data comprises:
segmenting the mammography image data, wherein the segmenting of the mammography image data includes segmenting a region of the extent of the cutout.

7. The method as claimed in claim 4, wherein the lack of attenuation caused by a lack of compression-plate material in the region of the cutout is determined based on a segmentation, and is determined in a segmented region of the cutout by applying a trained artificial neural network to the mammography image data of the cutout.

8. The method as claimed in claim 4, wherein the determining of the lack of attenuation caused by a lack of compression-plate material in the region of the cutout is performed directly by a trained holistic artificial neural network, wherein unsegmented mammography image data is used as input data.

9. The method as claimed in claim 1, wherein
the mammography image data includes spectral image data containing low-energy image data and high-energy image data, and
segmentation is performed an mammography image data of one of
the low-energy image data,
the high-energy image data, or
spectrally recombined image data.

10. The method as claimed in claim 1, wherein the corrected mammography image data is modified to at least partially compensate for a lack of attenuation caused by a convex distension of the breast into a region of an extent of the cutout.

11. The method as claimed in claim 10, wherein
the region of the extent of the cutout is determined by segmentation, and
the lack of attenuation resulting from the convex distension of the breast is determined by
a function curve adapted to grayscale values of the mammography image data in the region of the extent of the cutout, or
an artificial neural network.

12. An image data processing device, comprising:
an input interface configured to receive acquired mammography image data of a breast of a patient; and
a correction unit configured to determine corrected mammography image data based on the acquired mammography image data, wherein image effects of a cutout are determined in the acquired mammography image data, and the acquired mammography image data is modified to at least partially compensate for the image effects.

13. A mammography system comprising:
a scanner unit having a compression plate containing a cutout with vertical access for a biopsy needle, the scanner unit configured to acquire mammography image data of the breast of a patient;
a control device configured to control the scanner unit; and
the image data processing device as claimed in claim 12.

14. A non-transitory computer program product comprising commands that, on execution of the commands by a computer, cause the computer to perform the method as claimed in claim 1.

15. A non-transitory computer-readable storage medium comprising commands that, on execution by a computer, cause the computer to perform the method as claimed in claim 1.

16. The method of claim 5, wherein, in determining the corrected mammography image data, the constant offset is eliminated by grayscale values, in the corrected mammography image data at an edge of the cutout, adopting a same value outside and inside the cutout.

17. The method as claimed in claim 4, wherein the determining of at least one of the position or the extent of the cutout in the mammography image data based on the mammography image data comprises:
segmenting the mammography image data, wherein the segmenting of the mammography image data includes segmenting the region of the extent of the cutout.

18. The method as claimed in claim 5, wherein the determining of at least one of the position or the extent of the cutout in the mammography image data based on the mammography image data comprises:
segmenting the mammography image data, wherein the segmenting of the mammography image data includes segmenting the region of the extent of the cutout.

19. The method as claimed in claim 5, wherein the lack of attenuation caused by a lack of compression-plate material in the region of the cutout is determined based on a segmentation, and is determined in a segmented region of the cutout by applying a trained artificial neural network to the mammography image data of the cutout.

20. The method as claimed in claim 5, wherein the determining of the lack of attenuation caused by a lack of compression-plate material in the region of the cutout is performed directly by a trained holistic artificial neural network, wherein unsegmented mammography image data is used as input data.
